(19) United States
(12) Patent Application Publication
(10) Pub. No.: US 2013/0211241 A1
(43) Pub. Date: Aug. 15, 2013

(54) Local Coil System

(71) Applicants: Lars Lauer, Neunkirchen (DE); Dominik Paul, Bubenreuth (DE); Götz Welsch, Marloffstein (DE)

(72) Inventors: Lars Lauer, Neunkirchen (DE); Dominik Paul, Bubenreuth (DE); Götz Welsch, Marloffstein (DE)

(21) Appl. No.: 13/761,942

(22) Filed: Feb. 7, 2013

(30) Foreign Application Priority Data
Feb. 9, 2012 (DE) DE 102012201944.8

(51) Int. Cl. G01R 33/34; A61B 5/055; G01R 33/48

(52) U.S. Cl. CPC G01R 33/34092; G01R 33/48; A61B 5/055
USPC 600/422; 324/322; 324/309

(57) ABSTRACT

A local coil system having two loop coils is used for recording, for example, small joints in magnetic resonance tomography.

This application claims the priority benefit of German Patent Application No. DE 10 2012 201 944.8, filed Feb. 9, 2012, which is hereby incorporated by reference herein in its entirety.

### BACKGROUND

The present embodiments relate to methods and devices for recording, particularly for recording small joints (e.g., finger joints).

Magnetic-resonance tomography devices (MRTs) for examining objects or patients using magnetic-resonance tomography are known from, for instance, DE 10314215 B4.

### SUMMARY

The present embodiments aim to optimize MRT imaging, particularly MRT imaging of small joints.

### BRIEF DESCRIPTION OF THE DRAWINGS

FIG. 1 shows exemplary loop coils on a patient table;

FIG. 2 shows two exemplary loop coils on a patient's hand;

FIG. 3 is a cross-sectional view of an embodiment of an arrangement similar to the one shown in FIG. 2, with a finger of the patient's hand between the two loop coils; and

FIG. 4 shows one embodiment of an MRT system.

### DETAILED DESCRIPTION OF THE DRAWINGS

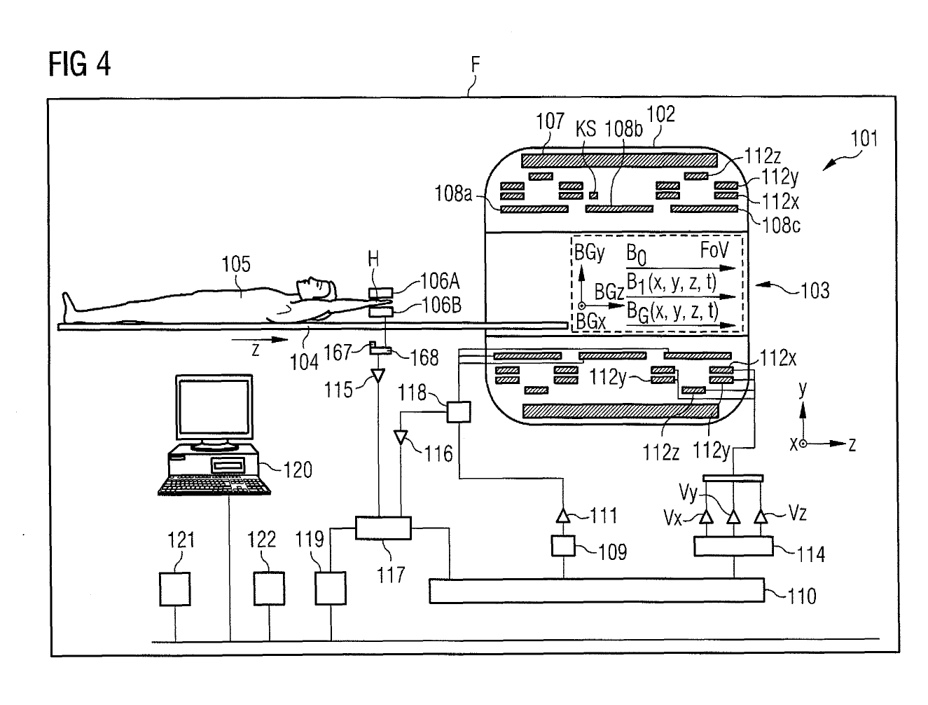

FIG. 4 shows an imaging magnetic-resonance tomography (MRT) device 101 (e.g., located in a shielded room or Faraday cage F) having a whole-body coil 102. The whole-body coil 102 has a tubular space 103 into which a patient table 104 along with a body of, for instance, an object 105 to be examined (e.g., a patient), with or without local coil arrangement 106, may be moved in the direction of arrow z for generating recordings of the object 105 by or using an imaging method. Arranged on the patient is a local coil arrangement 106, which may be used to generate recordings of a partial region of the body 105 in a local region (referred to also as a field of view or FoV) of the MRT. Signals of local coil arrangement 106 may be evaluated (e.g., converted into images, stored, or displayed) by an evaluation device, such as the evaluation device 168, 115, 117, 119, 120, or 121, that belongs to the MRT device 101 and may be connected to the local coil arrangement 106 via, for example, a coaxial cable or a radio link (e.g., the radio link 167).

In order to examine a body 105 (e.g., an object to be examined or a patient) with magnetic-resonance imaging using the MRT device 101, various magnetic fields, which are precisely mutually coordinated with respect to their temporal and spatial characteristics, are radiated onto the body 105. A powerful magnet (e.g., a cryomagnet 107) in a measuring cabin having, for example, tunnel-shaped opening 103 generates a powerful static main magnetic field B₀ (e.g., measuring 0.2 to 3 Tesla, or more than 3 Tesla). The body 105 to be examined is moved and positioned on the patient table 104 into a region of main magnetic field B₀ that is roughly homogeneous within the field of view. The nuclear spins of atomic nuclei of the body 105 are excited via magnetic high-frequency exciting pulses B1 (x, y, z, t) radiated into said nuclei via a high-frequency antenna (and/or a local coil arrangement), which is shown here in simplified form as a body coil 108 (e.g., with parts 108a, 108b, 108c). High-frequency exciting pulses are generated by, for example, a pulse-generating unit 109 controlled by a pulse-sequence control unit 110. The pulses are routed to the high-frequency antenna 108 after being amplified by a high-frequency amplifier 111. The high-frequency system shown in FIG. 4 is exemplary. In other embodiments, more than one pulse-generating unit 109, more than one high-frequency amplifier 111, a plurality of high-frequency antennas 108 a, b, c, or combinations thereof, may be employed in the MRT device 101.

The MRT device 101 further includes gradient coils 112x, 112y, 112z by which magnetic gradient fields B_{G} (x, y, z, t) are radiated for selective layer exciting and for spatially encoding the measuring signal during measuring. The gradient coils 112x, 112y, 112z are controlled by a gradient-coil control unit 114, which, like the pulse-generating unit 109, is connected to pulse-sequence control unit 110.

Signals emitted by the excited nuclear spins of the atomic nuclei in the object being examined are received by the body coil 108 and/or at least one local coil arrangement 106, amplified by assigned high-frequency pre-amplifiers 116, and further processed and digitized by a receiving unit 117. The recorded measurement data is digitized and stored in a k-space matrix in the form of complex numerical values. An associated MR image may be reconstructed from the value-containing k-space matrix by, for example, a multidimensional Fourier transformation.

For a coil that may be operated in both the transmitting and receiving mode, such as, for instance, the body coil 108 or a local coil 106, correct signal forwarding is controlled by an upstream diplexer 118.

An image-processing unit 119 generates an image from the measurement data. The image is displayed to a user on a control console 120 and/or stored in a storage unit 121. A central computer unit 122 controls the individual system components.

In MRT, images having a high signal-to-noise ratio (SNR) are generally recorded using local-coil arrays, which are antenna systems that are positioned in the immediate vicinity on (e.g., anteriorly), under (e.g., posteriorly), against, or in the body 105. During a magnetic resonance (MR) measurement, in the local coil's individual antennas the excited nuclei induce a voltage, which may then be amplified by a low-noise pre-amplifier and forwarded to the receiving electronic components. High-field systems (e.g., 1.5-12 Tesla or more) are also employed to improve the signal-to-noise ratio, particularly for high-resolution images. If a number of individual antennas that may be connected to an MR receiving system exceeds a number of receivers, a switch matrix (referred to also as RCCS) may, for example, be installed between the receiving antennas and receivers. The switch matrix will route the currently active receiving channels (usually the ones presently in the magnet's field of view) to the receivers. As such, more coil elements may be connected than there are receivers available, because with respect to, whole-body coverage, for example, the only coils that need to be read out are those located in the magnet's FoV, or, in some embodiments, located in the homogeneity volume.

The local coil arrangement 106 is, for example, an antenna system that may include, for example, one or more antenna elements (e.g., coil elements). The antenna elements may be or include, for example, loop antennas, a butterfly, flexible coils, or saddle coils. A local coil arrangement includes, for instance, coil elements, a pre-amplifier, other electronic components (baluns etc.), a housing, support, and, in some embodiments, a plug-terminated cable that connects the local coil arrangement to the MRT system. A receiver 168 mounted on the system side filters and digitizes a signal received from a local coil 106 (e.g., received by radio) and passes the data on to a digital signal-processing device. From the data obtained from a measurement, the signal-processing device may derive an image or spectrum and make the image or spectrum available to the user for, for example, diagnostic reasons (e.g., diagnosis by the user) and/or storing.

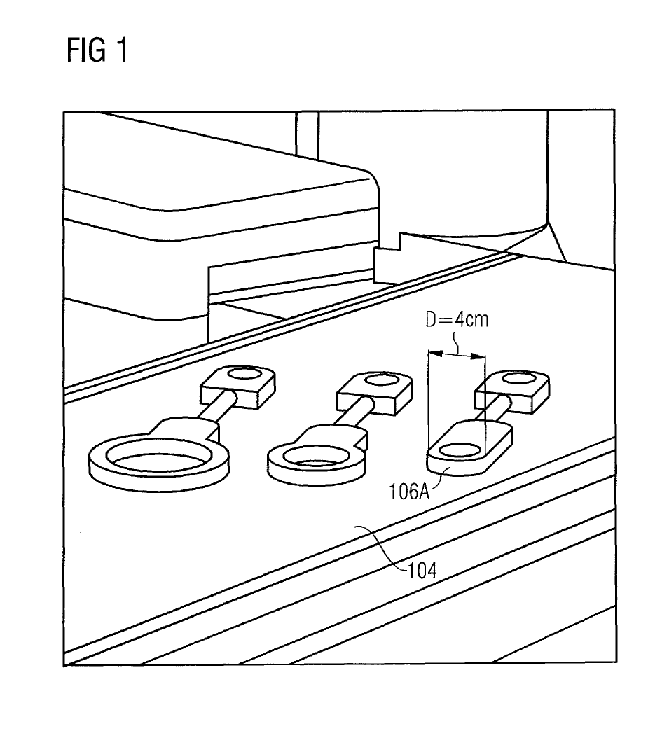

FIGS. 1-3 show embodiments of coil systems 106 that each include two local coils 106A, 106B.

Only a small spatial region is of particular interest when recording small joints G (such as, for example, an MCP=metacarpophalangeal joint=part of the proximal phalanx of the finger, or a PIP=proximal interphalangeal joint=a joint in the center of the finger, or other joints).

In one embodiment, the two local coils 106A, 106B are, for example, loop coils (e.g., local coils having a round recess in their center, local coils having a circular antenna).

FIG. 1 shows, for example, three loop coils lying on a patient table 104. The loop coils may have a diameter D (e.g., external diameter, clear internal diameter of a recess Aus, or antenna diameter) of up to six cm, or, in some embodiments, up to 4 cm (which may be a standard coil for MRT systems). In FIG. 1, one loop coil 106A has a diameter of 4-cm diameter. The coil system 106 includes two such small loop coils 106A, 106B.

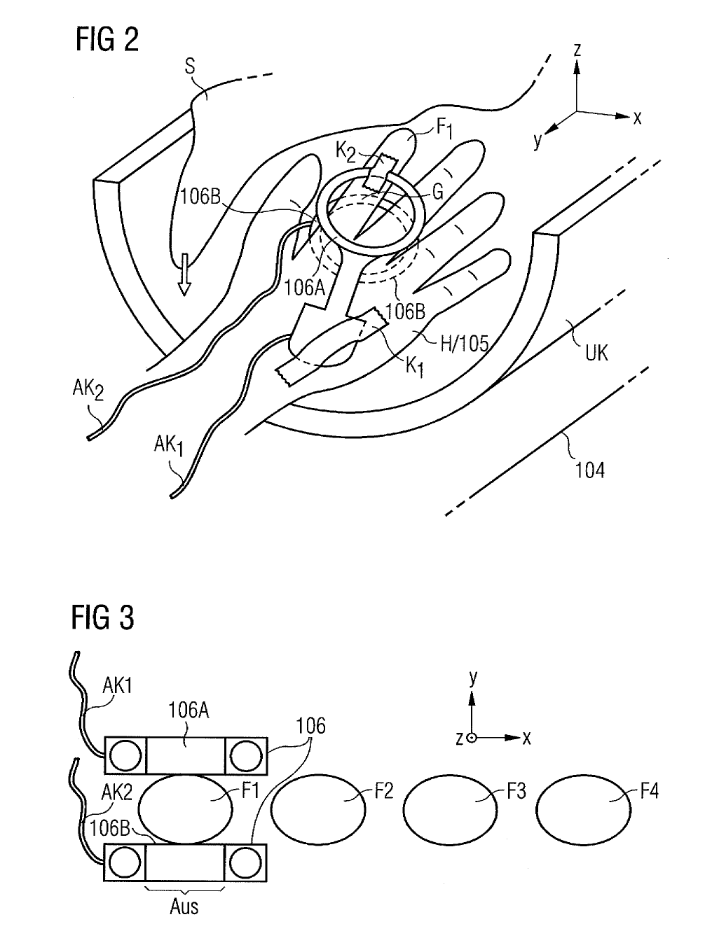

FIG. 2 shows a simultaneous usage of two loop coils 106A, 106B each having a diameter D of 4 cm and having connector cables AK1, AK2, or a radio link. The loop coils 106A, 106B have been suitably fitted around the anatomy H, F1, G being examined using MRI imaging, such that a spatially limited MRT-imaging region having a high spatial resolution and/or a high signal intensity in the MRT image recording may be provided.

In MCP recordings, for example, one loop coil may be positioned on the upper side of a hand H of the patient 105 and the other loop coil may be positioned on the inside of the hand H, with both local coils positioned over the relevant joint/joints.

In one embodiment, as shown in FIG. 2, one loop coil 106A may be positioned on the upper side of the hand H and/or the top side of at least one finger F1 of the hand H of the patient 105 and the other loop coil 106B may be positioned on the inside of the hand H and/or the underside of at least one finger F1. The loop coils 106A, 106B may be fixed into place using, for example, adhesive tape K1, K2. The hand H, which is resting, may be secured into position using, for example, plasters or a cushion S, on what is, in this embodiment, a U-shaped support UK on the patient table 104.

FIG. 3 is a cross-sectional view of a coil system 106 similar to the one shown in FIG. 2, with one finger F1 of fingers F1, F2, F3, F4 of one hand H being located between the two loop coils 106A, 106B, such that at least one MRT image of a joint G of finger F1 may be produced through MRT imaging using the loop coils 106A, 106B and the MRT device 101.

In FIG. 3, the loop coils 106A, 106B are not mutually connected (e.g., not fixed to each other or secured to each other) before or during MRT imaging, such that the two loop coils 106A, 106B may be used independently and may be individually located and secured (using, for example, the adhesive tape AK1, AK2) in region G, F1, H, or combinations thereof, of the joint G being examined.

Using two loop coils may permit, for example, a parallel imaging technique to be employed (such as Grappe/Grappa or Sense). Compared with, for instance, a flexible coil, a recording by or using a local coil system that includes the two loop coils 106A, 106B may provide a higher signal intensity, higher resolution, and/or a shorter measuring time.

In one embodiment, two loop coils may be used in parallel. The two loop coils may be suitably positioned on the region G to be examined for making an MRT recording of small joints G. For example, one loop coil may be placed over the recording region and/or joint G to be recorded and one loop coil may be placed thereunder. The loop coils may be secured upon the joint during an MRT examination by a clamp, adhesive tape, or other retaining means or aid(s).

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A local coil system for an imaging magnetic resonance tomography (MRT) system, the local coil system comprising:
two loop coils.

2. The local coil system as claimed in claim 1, wherein one or more of the two loop coils have a diameter of less than six cm.

3. The local coil system as claimed in claim 1, wherein one or more of the two loop coils have a diameter of less than four cm.

4. The local coil system as claimed in claim 1, wherein the two loop coils each have a circular antenna surrounding a recess in the respective loop coils.

5. The local coil system as claimed in claim 4, wherein one or more of the two loop coils have a diameter of less than six cm.

6. The local coil system as claimed in claim 4, wherein one or more of the two loop coils have a diameter of less than four cm.

7. The local coil system as claimed in claim 1, wherein the local coil system is a joint-MRT-recording local coil system configured to record a small joint of an object to be examined, the small joint measuring less than three cm in diameter.

8. The local coil system as claimed in claim 1, wherein the local coil system is a joint-MRT-recording local coil system configured to record a finger joint of an object to be examined.

9. The local coil system as claimed in claim 1, wherein in the local coil system is an metacarpophalangeal (MCP)-joint local coil system configured for MRT imaging on an MCP finger joint.

10. The local coil system as claimed in claim 1, wherein during MRT imaging the two loop coils of the local coil system are arranged such that a region of an object to be examined is located there between.

11. The local coil system as claimed in claim 1, wherein during MRT imaging the two loop coils of the local coil system are arranged such that a joint of an object to be examined is located there between.

12. The local coil system as claimed in claim 1, wherein during MRT imaging the two loop coils of the local coil system are located on a joint, a finger, a hand, or combinations thereof.

13. The local coil system as claimed in claim 1, wherein during MRT imaging the two loop coils of the local coil system are secured to a joint, a finger, a hand, or combinations thereof, of an object.

14. The local coil system as claimed in claim 1, wherein the two loop coils are not secured to each other before, during, or before and during MRT imaging.

15. The local coil system as claimed in claim 1, wherein the two loop coils are not secured to each other before and during MRT imaging.

16. A method comprising:
performing MRT imaging on a joint of an object to be examined using a local coil system having two loop coils.

17. The method as claimed in claim 16, wherein performing MRT imaging comprises performing the MRT imaging using the local coil system having one or more loop coils that have a diameter of less than six cm.

18. The method as claimed in claim 16, wherein performing MRT imaging comprises performing the MRT imaging using the local coil system having one or more loop coils that have a diameter of less than four cm.

19. The method as claimed in claim 16, wherein performing comprises performing with the two loop coils each having a circular antenna.

20. The method as claimed in claim 16, wherein performing MRT imaging comprises performing MRT imaging on a small joint to be examined using a joint-recording coil system having the two loop coils, the small joint measuring less than three cm in diameter.

21. The method as claimed in claim 16, wherein performing MRT imaging comprises performing the MRT imaging on a metacarpophalangeal (MCP) finger joint to be examined using a MCP-joint local coil system having the two loop coils.

22. The method as claimed in claim 16, wherein performing the MRT imaging comprises arranging the two loop coils of the local coil system such that a region to be examined of the object is located there between.

23. The method as claimed in claim 16, wherein performing the MRT imaging comprises arranging the two loop coils of the local coil system such that a joint to be examined of the object is located between them.

24. The method as claimed in claim 16, wherein performing the MRT imaging comprises positioning the two loop coils on and securing the two loop coils to a joint, a finger, a hand, or combinations thereof, of the object.

25. The method as claimed in claim 16, wherein performing the MRT imaging comprises positioning one of the two loop coils over a joint, a hand, or both the joint and the hand of the object, and positioning the other one of the two loop coils under the joint, the hand, or both the joint and the hand of the object.
